(19) United States
(12) Patent Application Publication
(10) Pub. No.: US 2008/0269659 A1
(43) Pub. Date: Oct. 30, 2008

(54) Hemostatic Bandage

(76) Inventors: Patrick J. Bergin, Eugene, OR (US); Jeffrey P. Wensel, Eugene, OR (US)

Correspondence Address: Finnegan, Henderson, Farabow, Garrett & Dunner,LLP;ATTN: Les Bookoff, 901 New York Avenue, NW, Washington, DC, 20001-4413, US

(21) Appl. No.: 11/862,187

(22) Filed: Sep. 26, 2007

Related U.S. Application Data
(63) Continuation-in-part of application No. 11/245,956, filed on Oct. 7, 2005.
Application No. 11/245,956 is a continuation-in-part of application No. 11/332,784, filed on Jan. 12, 2006.
Application No. 11/332,784 is a continuation-in-part of application No. 11/671,448, filed on Feb. 5, 2007.
(60) Provisional application No. 60/827,055, filed on Sep. 26, 2006.
(60) Provisional application No. 60/693,706, filed on Jun. 24, 2005.
(60) Provisional application No. 60/688,510, filed on Jun. 7, 2005.

(51) Int. Cl. A61F 13/00; A61B 17/12; A61L 15/44; A61F 13/10

(52) U.S. Cl. 602/49; 602/79; 606/213; 602/62

(57) ABSTRACT

Some embodiments of the invention provide an apparatus for achieving hemostasis in a puncture tract in a patient (e.g., a human or animal). Such a tract might have been created during a medical procedure or operation. Alternatively, the tract might be a result of a traumatic injury (e.g., injury that occurred outside of a hospital) that created a traumatic wound, such as a bullet wound, shrapnel or knife puncture. The puncture typically extends from the epidermis to the vasculature and/or internal organs in a living organism. In some embodiments, the apparatus includes (1) a bandage for inserting at least partially into a puncture tract to achieve hemostasis, and (2) a strap for maintaining the bandage on a part of a patient (e.g., a part of a human or animal such as appendage, torso, extremity, etc.). The strap maintains the bandage within the puncture tract at a particular pressure in some embodiments. The bandage and strap are removed from the patient after a time period (e.g., once hemostasis is achieved).

### CLAIM OF BENEFIT

This application claims the benefit of U.S. Provisional Application 60/827,055, filed on Sep. 26, 2006. This application is a continuation-in-part of U.S. patent application Ser. No. 11/245,956, filed on Oct. 7, 2005, is a continuation-in-part of U.S. patent application Ser. No. 11/332,784, filed on Jan. 12, 2006, and is a continuation-in-part of U.S. patent application Ser. No. 11/671,448, filed Feb. 5, 2007. All of these non-provisional applications claim the benefit of U.S. Provisional Application 60/693,706, filed on Jun. 24, 2005, and claim the benefit of U.S. Provisional Application 60/688,510, filed on Jun. 7, 2005. The nonprovisional application Ser. Nos. 11/245,956, 11/332,784, and 11/671,448 and the provisional application 60/827,055 are incorporated herein by reference.

### BACKGROUND OF THE INVENTION

Numerous medical diagnostic and therapeutic procedures require access to the internal organs of a living organism. Some of these procedures can be performed without traditional surgical incisions by utilizing catheter-based apparatus to enter blood vessels. Usually, catheter-based apparatus require a needle to be inserted through the skin and directed into a blood vessel. This provides a conduit for extending a metal or polymer guide wire through the needle and into the vasculature. After positioning the guide wire in the conduit, the needle can be removed and replaced with a hollow tube or catheter directed over the guide wire into the blood vessel. The tube or catheter provides access for administration of certain substances and/or for passage of additional equipment that will be used to perform manipulations within the vasculature or within other organ systems accessible through the vasculature.

To prevent bleeding upon completion of a catheter-based intravascular procedure, the catheter must be removed and the puncture site sealed. In the low-pressure environment of the venous system, a small needle puncture is readily sealed by the brief application of pressure to the site and application of a light dressing, such as a bandage. This method is widely utilized after needle stick procedures such as blood drawings.

However, when punctures are created with larger caliber apparatus (such as catheters) in the high-pressure environment of arteries, the puncture created will not readily seal with the application of brief pressure. Prolonged external pressure may be required for fifteen to thirty minutes and may lead to substantial discomfort at the puncture site for the patient and/or a significant failure rate with late bleeding and hematoma formation.

In the past, several methods have been proposed to address this problem. For instance, one prior apparatus utilizes a marker to indicate the position of the bandage with respect to the wound to be treated in order to position externally applied pressure at or near a puncture site. Another apparatus uses a pad which, when moistened by fluid from a wound, expands and exerts pressure against a wound.

Another apparatus utilizes laser energy directed through a balloon tipped catheter into the vascular tract and positioned just outside the outer wall of the blood vessel. The balloon is used to create a covering for the vascular puncture. The laser is used to create a laser “weld” or seal in the adjacent tissue.

Another apparatus uses both a balloon tipped catheter and an absorbable plug. The plug is used to occlude the vascular access tract and provide hemostasis. The balloon tipped catheter serves as a positioning anchor for antegrade insertion of the vascular plug and must be removed from the patient after plug deployment.

Yet another apparatus uses a balloon tipped catheter arranged so as to pass into the vascular lumen by means of the extant access sheath. After this procedure, it is withdrawn from the intraluminal side of the blood vessel puncture to provide temporary hemostasis. A pro-coagulant slurry is then injected into the vascular access tract to promote coagulation. During this time, the balloon tipped catheter remains inflated. After a suitable period of time necessary to promote blood coagulation, the balloon tipped catheter is deflated and withdrawn from the access tract.

Each of these approaches has its own unique set of shortcomings. The prior apparatus lack both a mechanism for precise positioning of a pressure-generating component against a puncture tract and a structure designed to optimize the pressure that is to be applied to such a site. Therefore, there is a need in the art for an apparatus that hemostatically closes a vascular puncture site without leaving a hematoma within the puncture tract, while minimizing patient discomfort. Ideally, such an apparatus would quickly, painlessly and reliably achieve hemostasis upon withdrawal of vascular catheters. Consequently, such an apparatus would reduce patient discomfort, staff time and the unfavorable failure rate associated with vascular hemostasis and the risk of hematoma formation.

### SUMMARY OF THE INVENTION

Some embodiments of the invention provide an apparatus for achieving hemostasis in a puncture tract in a patient (e.g., a human or animal). Such a tract might have been created during a medical procedure or operation. Alternatively, the tract might be a result of a traumatic injury (e.g., injury that occurred outside of a hospital) that created a traumatic wound, such as a bullet wound, shrapnel or knife puncture. The puncture typically extends from the epidermis to the vasculature and/or internal organs in a living organism.

In some embodiments, the apparatus includes (1) a bandage for inserting at least partially into a puncture tract to achieve hemostasis, and (2) a strap for maintaining the bandage on a part of a patient (e.g., a part of a human or animal such as appendage, torso, extremity, etc.). The strap maintains the bandage within the puncture tract at a particular pressure in some embodiments. The bandage and strap are removed from the patient after a time period (e.g., once hemostasis is achieved).

Some embodiments of the invention also provide a method for achieving hemostasis. The method inserts a bandage at least partially into a puncture tract. The method uses a strap that is coupled to the bandage to maintain the bandage on a part of a body that includes the puncture tract. In some embodiments, the method removes the bandage from the puncture tract after a time period (e.g., once hemostasis has been achieved).

In some embodiments, the bandage is composed of polymeric foam. In some embodiments, the polymeric foam is hydrophilic polyurethane foam. The bandage can have many shapes, but typically has a shape that facilitates its insertion into the puncture tract. For instance, in some embodiments, the bandage has a tapered tip.

Also, in some embodiments, the bandage includes or is coated with one or more materials (e.g., Chitosan) that are designed to promote coagulation and thereby achieve hemostasis. For example, in some embodiments, the bandage includes, is coated with, or is entirely composed of Chitosan. In some embodiments, the Chitosan may be incorporated on and into the foam bandage by means of dipping the bandage into a Chitosan solution. In some embodiments, the foam bandage is lyophilized after dipping in the Chitosan solution.

In some embodiments, the strap partially wraps around a part of the patient's body. In other embodiments, the strap completely wraps around a part of the patient's body (i.e., a first portion of the strap overlaps with a second portion of the strap). In still other embodiments, the strap completely wraps around a first part (e.g., an arm) of the patient, but can only partially wrap around a second part (e.g., a thigh) of the patient. In some embodiments, the strap is adjustable so that it can affix to different parts of a patient's body or to the same part of different patients who have different sizes. In some embodiments, the strap has an adhesive portion that affixes the strap to the patient's body.

In some of these embodiments, the adjustable strap is a wristband that is wrapped around the patient's wrist, after the completion of an operation that uses the radial artery (which is an artery that passes through the patient's wrist). In other words, the strap of some embodiments is a wristband designed for a radial artery application. In these embodiments, the bandage is for achieving hemostasis of a radial artery in a wrist. In other embodiments, the strap is an arm band and the bandage is for achieving hemostasis of a brachial artery in an arm.

In different embodiments, the bandage is inserted into the puncture tract at different depths. In some embodiments, the puncture tract typically extends from the epidermis to a vascular puncture in a living organism. For instance, the bandage might be inserted into the epidermis layer, the dermis layer, the subcutaneous tissue layer, etc. In yet other embodiments, the bandage is not inserted into the puncture tract, and is instead simply placed on the patient's skin (i.e., on the epidermis) over the puncture tract.

In some embodiments, the bandage is wire-guided into the puncture tract. In other words, some embodiments guide the adjustable strap and its associated bandage into or over the puncture tract by using a wire that is inserted into the puncture tract and that is threaded through a hole in the bandage and/or strap. The adjustable strap in other embodiments is not wire-guided. Instead, it is simply secured to the patient by visually placing its plug within or over the puncture tract.

As mentioned above, the strap and its associated bandage are removed after hemostasis has been achieved in some embodiments. In this manner, the strap and its associated bandage act as a disposable hemostatic apparatus. In these embodiments, the bandage is the component of the disposable bandage apparatus that is inserted into the puncture tract to achieve hemostasis. Accordingly, the bandage is also referred to below as a “plug” as it blocks the puncture tract.

### BRIEF DESCRIPTION OF THE DRAWINGS

The novel features of the invention are set forth in the appended claims. However, for purpose of explanation, several embodiments of the invention are set forth in the following Figures.

FIG. 1 illustrates a side elevation view showing in cross section, a hemostasis sheath placed over a guide wire within a blood vessel through the epidermis and subcutaneous tissue of a living being.

FIG. 2 illustrates a side elevation view showing in cross section, a guide wire in place with the hemostasis sheath removed.

FIG. 3 illustrates a side elevation view showing in cross section the hemostatic bandage being passed over the guide wire and into the puncture wound.

FIG. 4 illustrates a side elevation view showing in cross section, the occlusive feature of the bandage after being set in place with the guide wire removed and the hemostatic bandage secured within the puncture tract.

FIG. 5 illustrates a side elevation view of the component parts of the hemostatic bandage.

FIG. 6 illustrates an oblique, three-dimensional exploded view of the component parts of the hemostatic bandage.

FIG. 7 illustrates a side elevation view showing in cross section, a guide wire in place in the radial artery with the hemostasis sheath removed.

FIG. 8 illustrates a side elevation view showing in cross section, the radial artery hemostatic bandage being passed over the guide wire and into the puncture wound in the radial artery.

FIG. 9 illustrates a side elevation view showing in cross section, the radial artery hemostatic bandage in place with the guide wire removed and the hemostatic bandage secured within the puncture tract in the radial artery.

FIG. 10 illustrates an oblique three-dimensional view of the component parts of the radial artery hemostatic apparatus of some embodiments of the invention with the apparatus in place over the radial artery of the wrist.

FIG. 11 illustrates an oblique three-dimensional view of the component parts of the radial artery hemostatic apparatus of some embodiments of the invention.

### DETAILED DESCRIPTION OF THE INVENTION

In the following description, numerous details are set forth to provide a better understanding of the various embodiments of the invention. However, one of reasonable skill in the art will realize that the invention may be practiced without the use of the specific details presented herein. In some instances of describing the invention, well-known structures and apparatus may be shown in block diagram form to avoid obscuring the description of the invention with unnecessary detail. Therefore, the examples provided herein for clarification and understanding should not be read into and thereby limit the language of the claims.

Some embodiments of the invention provide an apparatus for achieving hemostasis in a puncture tract that is created during a medical procedure. The puncture typically extends from the epidermis to the vasculature in a living organism. In some embodiments, the apparatus includes (1) a bandage for subcutaneous placement within the puncture tract, and (2) a delivery mechanism for delivering and maintaining the bandage within the puncture tract until hemostasis is achieved.

In some embodiments, the delivery mechanism and its associated bandage are removed after hemostasis has been achieved. In this manner, the delivery mechanism and its associated bandage act as a disposable hemostatic apparatus. In these embodiments, the bandage is the component of the disposable bandage apparatus that is inserted into the puncture tract to achieve hemostasis. Accordingly, the bandage is also referred to below as a “plug” as it blocks the puncture tract.

In some embodiments, the bandage (i.e., plug) is part of an adjustable strap that is used to secure the bandage to a patient (e.g., a part of a human or animal such as appendage, torso, extremity, etc.) and maintain pressure until hemostasis has been achieved. For instance, in some embodiments, the apparatus includes (1) a bandage for inserting at least partially into a puncture tract to achieve hemostasis, and (2) a strap for maintaining the bandage on a part of a patient's body. The strap maintains the bandage within the puncture tract at a particular pressure in some embodiments. The bandage and strap are removed from the patient after a time period (e.g., once hemostasis is achieved).

In some embodiments, the bandage is composed of polymeric foam. In some embodiments, the polymeric foam is hydrophilic polyurethane foam. The bandage can have many shapes, but typically has a shape that facilitates its insertion into the puncture tract. For instance, in some embodiments, the bandage has a tapered tip.

Also, in some embodiments, the bandage includes or is coated with one or more materials (e.g., Chitosan) that are designed to promote coagulation and thereby achieve hemostasis. For example, in some embodiments, the bandage includes, is coated with, or is entirely composed of Chitosan. In some embodiments, the Chitosan may be incorporated on and into the foam bandage by means of dipping the bandage into a Chitosan solution. In some embodiments, the foam bandage is lyophilized after dipping in the Chitosan solution.

In some embodiments, the strap partially wraps around a part of the patient's body. In other embodiments, the strap completely wraps around a part of the patient's body (i.e., a first portion of the strap overlaps with a second portion of the strap). In still other embodiments, the strap completely wraps around a first part (e.g., an arm) of the patient, but can only partially wrap around a second part (e.g., a thigh) of the patient. In some embodiments, the strap is adjustable so that it can affix to different parts of a patient's body or to the same part of different patients who have different sizes. In some embodiments, the strap has an adhesive portion that affixes the strap to the patient's body.

In some of these embodiments, the adjustable strap is a wristband that is wrapped around the patient's wrist, after the completion of an operation that uses the radial artery (which is an artery that passes through the patient's wrist). In other words, the strap of some embodiments is a wristband designed for a radial artery application. In these embodiments, the bandage is for achieving hemostasis of a radial artery in a wrist. In other embodiments, the strap is an arm band and the bandage is for achieving hemostasis of a brachial artery in an arm.

In different embodiments, the bandage is inserted into the puncture tract at different depths. In some embodiments, the puncture typically extends from the epidermis to a vascular puncture in a living organism. For instance, the bandage might be inserted into the epidermis layer, the dermis layer, the subcutaneous tissue layer, etc. In yet other embodiments, the bandage is not inserted into the puncture tract, and is instead simply placed on the patient's skin (i.e., on the epidermis) over the puncture tract.

In some embodiments, the bandage is wire-guided into the puncture tract. In other words, some embodiments guide the adjustable strap and its associated bandage into or over the puncture tract by using a wire that is inserted into the puncture tract and that is threaded through a hole in the bandage and/or strap. The adjustable strap in other embodiments is not wire-guided. Instead, it is simply secured to the patient by visually placing its plug within or over the puncture tract.

Several more detailed embodiments of the invention are discussed in Sections III-IV. These embodiments provide hemostatic bandages and wire-guided bandage delivery systems. Before discussing these embodiments, it is helpful to understand relevant terminology and some environments in which the hemostatic bandage and its associated delivery system are used. Therefore, Section I presents relevant terminology. Section II provides an overview of intravascular procedures, which are one type of procedure in which the invention can be used.

### I. Terms and Terminology

An opening in the skin is called a percutaneous opening because it passes through the skin. The skin can be described in terms of the epidermis skin layer, dermis skin layer, and subcutaneous tissue skin layer. The hole from the percutaneous opening to the blood vessel is the puncture tract or access tract. The terms puncture tract and access tract are used interchangeably throughout the specification. In some embodiments, the tract can be created during a medical procedure or operation.

The opening in the blood vessel wall is a vascular puncture or vascular opening. The open space within the blood vessel is called the vascular lumen. As used in the following discussion, a “lumen” is an opening, such as the cavity of a tubular organ or the bore of a tube (as of a hollow needle or catheter). The term “bandage” is used generically to refer to an apparatus that assists in achieving hemostasis of a wound. In some embodiments, the bandage is a plug.

### II. An Exemplary Intravascular Procedure

Some embodiments of the invention have particular utility when utilized in conjunction with intravascular procedures. Today, many physicians, such as radiologists and cardiologists, perform intravascular procedures. Examples of intravascular procedures include angiography, angioplasty, vascular stenting and stent graft placement, arterial thrombectomy, arterial embolization, intra-arterial drug administration, etc. These procedures normally involve the insertion of a hollow needle (e.g., an 18 gauge thin walled needle) through the skin. The needle is advanced through the body tissue overlying a blood vessel and continued through the proximal side of the vascular wall until the distal tip of the needle enters the vascular lumen. A brisk return of blood through the needle hub signals entry of the needle into the vascular lumen.

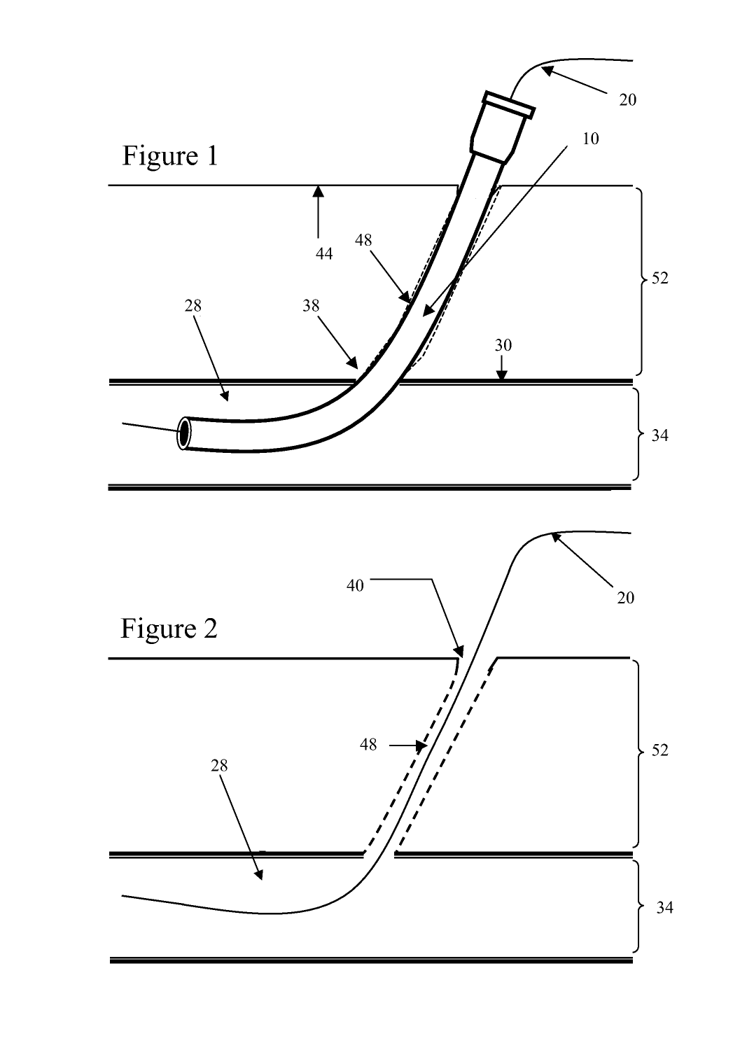

FIGS. 1 and 2 illustrate an exemplary intravascular procedure that commonly uses an access sheath 10 placed in the access tract 48 to facilitate entry into the vascular lumen 34 by diagnostic and therapeutic tools. FIG. 1 illustrates the hemostasis access sheath 10 threaded onto a guide wire 20 and placed within the access tract 48.

To install the access sheath 10, the operator first creates an access path to the blood vessel 28 by cutting a percutaneous opening 40 in the epidermal skin layer 44 at a point that is favorable to accessing the blood vessel 28. A needle (or other cutting tool) is typically advanced through a percutaneous opening 40, an epidermal skin layer 44, a dermal skin layer, a subcutaneous tissue skin layer 52 and a vascular wall 30. It continues through the vascular wall 30 (creating a vascular puncture 38) and into a vascular lumen 34 of a blood vessel 28. This creates the access tract 48.

After creating the access tract 48, the operator typically threads a guide wire 20 longitudinally through the needle. After positioning the guide wire 20 within the access tract 48, the needle may be removed while maintaining the guide wire 20 in position. Normally, an access sheath 10 is later placed within the access tract 48 to prevent the tract 48 from closing during the procedure. The access sheath 10 is typically threaded onto the guide wire 20 and inserted into the access tract 48, using the guide wire 20 to precisely position the sheath 10 into place. When positioned at its final location, one end of the sheath 10 is within the vascular lumen 34 while the opposing end is outside of the organism. Once the access sheath 10 is in place, other apparatus and/or materials can pass through the access sheath 10 and advance into the blood vessel 28 to the area of interest within the body, in order to perform the intravascular procedure.

Upon completion of the intravascular procedure, the catheters and other apparatus used in the procedure are removed from the blood vessel 28 and/or the puncture tract 48. This is generally followed by the removal of the sheath 10 over the guide wire 20, leaving the guide wire 20 in place within the access tract 48 and leaving the access tract 48 open. FIG. 2 presents a longitudinal cross-sectional side view of the access tract 48 with the guide wire 20 in place after the removal of the access sheath 10.

The removal of tools from the access tract 48 causes the access tract 48 to gradually close upon any objects remaining within the access tract 48. If hemostasis is not quickly attained, vigorous bleeding can occur. Therefore, the vascular puncture 38 and the access tract 48 must be sealed as quickly and as efficiently as possible. One method of doing so uses a hemostatic wire guided bandage delivery and placement apparatus of some embodiments of the invention.

### III. Hemostatic Bandage and Wire-Guided Delivery System for Delivering the Hemostatic Bandage in a Puncture Tract

Some embodiments provide a hemostatic bandage for achieving hemostasis in a puncture tract that is created during a medical procedure. Some embodiments also include a wire-guided delivery mechanism for delivering the bandage into the puncture tract and for maintaining the bandage in the puncture tract until hemostasis is achieved. In some embodiments, the mechanism not only positions the bandage, but also occludes the opening of the puncture tract. Some of the embodiments described below of a hemostatic wire guided bandage delivery and placement apparatus achieve hemostasis at or near a vascular puncture site in a human patient after an intravascular procedure. However, the apparatus' construction and use also have widespread applicability in other settings, e.g., for non-intravascular procedure or for non-human patients (i.e., other living organisms).

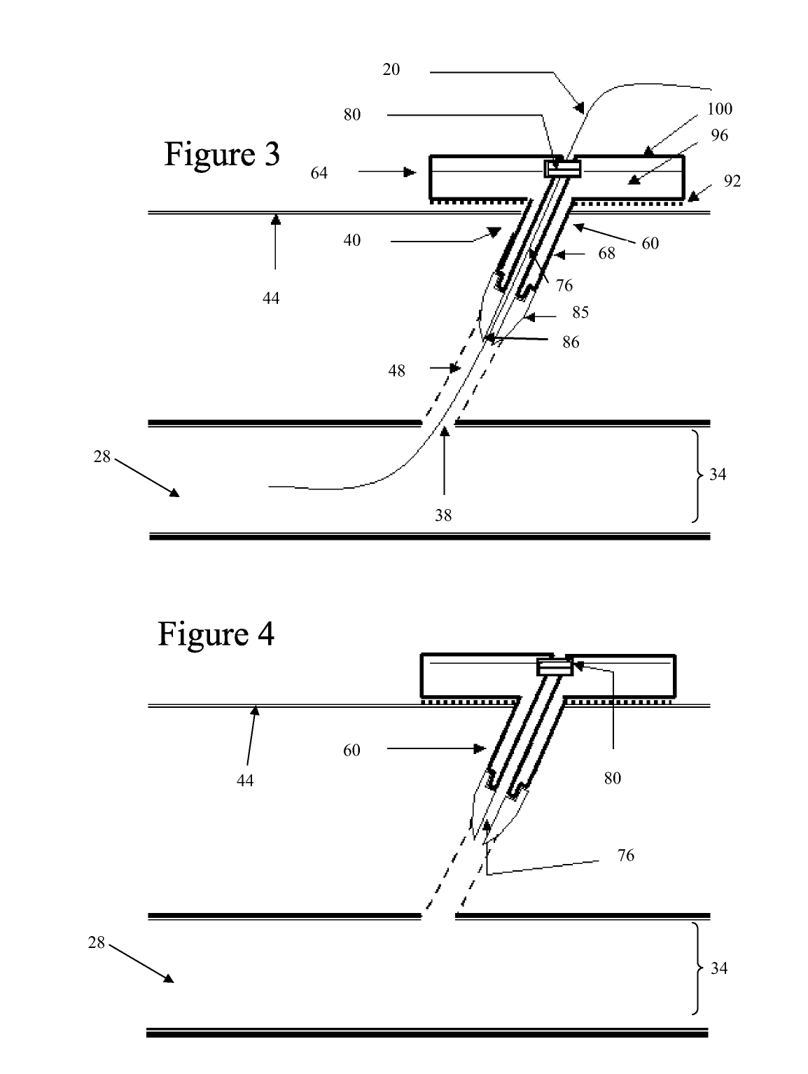

FIGS. 3 through 5 illustrate a hemostatic apparatus 60 of some embodiments of the invention. This apparatus includes a hemostatic bandage and its associated wire guided delivery apparatus. As shown in FIG. 3, the hemostatic apparatus 60 includes (1) a cover pad 64, (2) a stem 68 affixed to the cover pad 64 and extending at a angle downwards from the bottom side of the cover pad 64, (3) a bandage 85 attached to the distal end of the stem 68, and (4) a central lumen 76 defined from the top of the cover pad downwards through the center of the stem 68 and through the center of the bandage 85. As shown in this figure, the cover pad includes a hemostatic valve 80.

As shown in FIG. 3, the hemostatic apparatus 60 positions the bandage 85 within the puncture tract 48 (e.g., subcutaneously within the tract) to provide hemostasis within a puncture tract 48. In use, the cover pad 64 of the hemostatic apparatus 60 can cover and/or occlude the access tract 48 percutaneously. The cover pad's hemostatic valve 80 prevents blood from flowing back through the central lumen and out of the patient, while allowing for the passage of the guide wire 20 through the central lumen.

The stem 68 positions the bandage 85 within the access tract 48 to achieve hemostasis. As mentioned above, the stem can extend downwards at an angle from the bottom side of the cover pad 64. This angle corresponds to the angle of the puncture tract 48. In some embodiments, the angle at which the stem 68 extends downwards from the cover pad 64 is adjustable to match the angle of the puncture tract 48.

While FIG. 3 presents the guide wire 20 threaded through the hemostatic apparatus 60, FIG. 4 presents the hemostatic apparatus 60 after the guide wire 20 has been removed. The guide wire 20 is used to properly guide the bandage 85 as the hemostatic apparatus 60 is advanced into the access tract 48. After the apparatus 60 is in place, the guide wire 20 may be removed, as shown in FIG. 4. Its removal from the access tract 48 causes the access tract to gradually close further.

The cover pad 64, hemostatic valve 80, a stem 68 and bandage 85 of the hemostatic apparatus 60 are discussed in detail in Section A, immediately below. This discussion is followed in Section B by a description of how the hemostatic apparatus 60 is used in some embodiments to place a hemostasis bandage subcutaneously within a puncture tract 48.

A. Components of a Bandage Delivery and Placement Apparatus

1. The Cover Pad

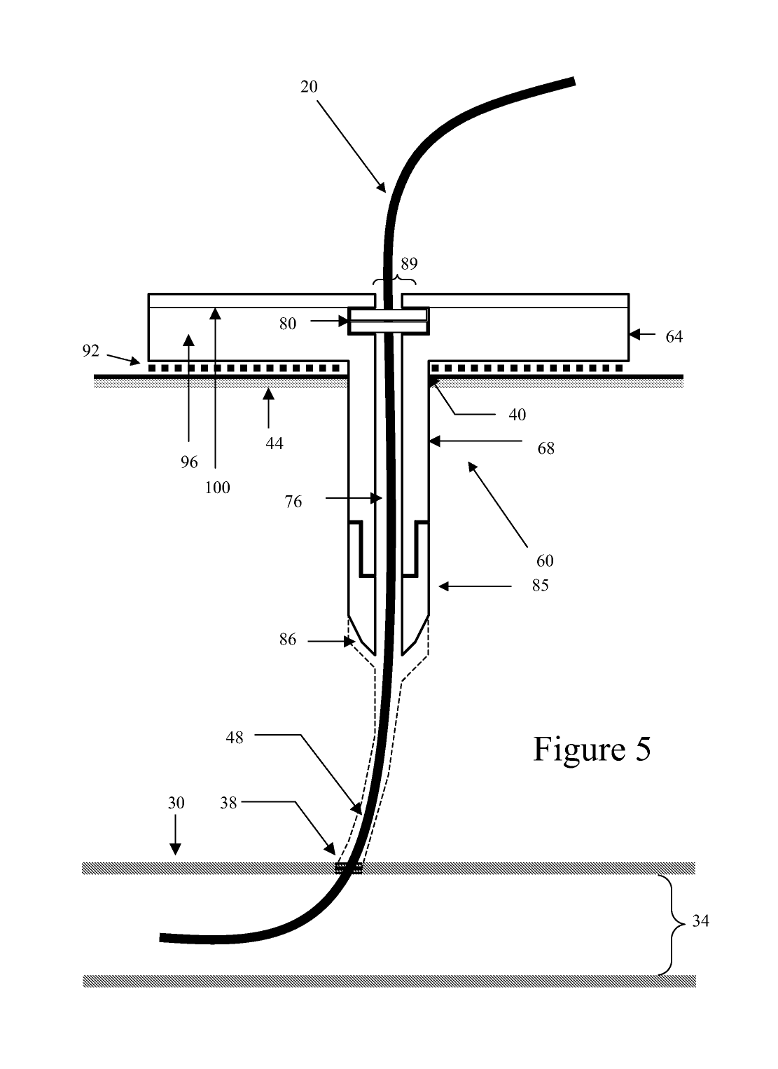

In some embodiments, the cover pad 64 provides a mechanism (1) to act as a handle for holding and maneuvering the apparatus 60, (2) to push or pull the stem 68 into or out of the access tract 48, (3) to occlude the percutaneous opening 40, and (4) to affix the hemostatic apparatus 60 to the epidermal layer 44 during recovery. FIG. 5 presents a more detailed view of the hemostatic apparatus 60. As shown in this figure, the hemostatic apparatus 60 in some embodiments includes a multi-layered cover pad 64. The layers include a first adhesive layer 92, a second central layer 96 and a third surface layer 100. The cover pad in some embodiments includes a fourth layer (not shown in FIG. 5) that covers the first adhesive layer 92 as further described below. Although FIG. 5 shows a particular multi-layered cover pad, a person skilled in the art will realize that the cover pad 64 in other embodiments might be constructed differently (e.g., with more or less layers).

As mentioned above, the first layer 92 of the cover pad 64 in some embodiments is an adhesive layer that is applied to the bottom side of the second central layer 96 of the cover pad 64. The first adhesive layer 92 is covered by a fourth layer (not shown) when the bandage has not been deployed. The fourth layer protects the adhesive layer 92 from degradation before the bandage 85 has been deployed. As further described below, the fourth layer is removed from the first layer 92 when the bandage is being deployed, in order to enable the first layer 92 to affix the apparatus 60 to the patient's skin during the procedure.

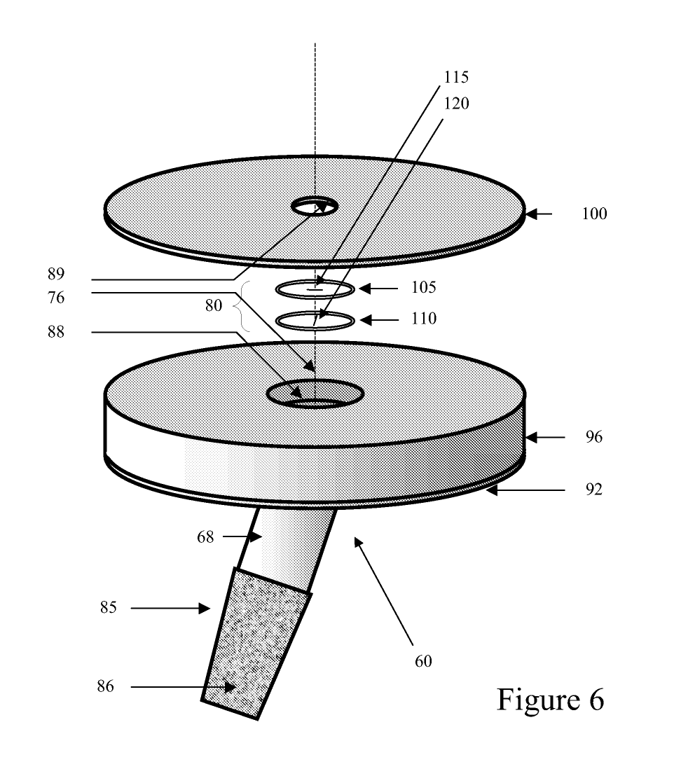

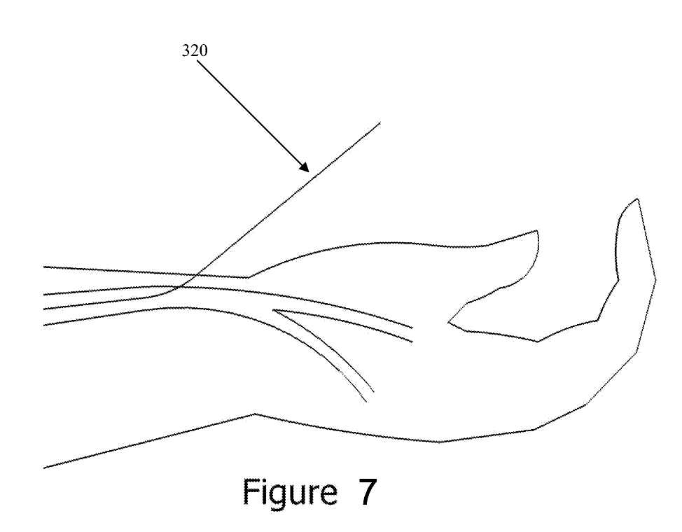

As illustrated in FIG. 6, the second layer 96 has a second layer lumen 88 defined about the central lumen 76, which passes through the second layer 96. The hemostatic valve 80 is seated in the second layer lumen 88, which is larger than, and concentric to, the central lumen 76 and is shaped to receive the valve 80. With the valve 80 seated in the second layer lumen 88, the third layer 100 covers the second layer 96 (including the valve 80) to immobilize the valve 80 within the second layer lumen 88. The third layer 100 contains a third layer lumen 89 that is concentric (i.e., is defined about the same axis) to the central lumen 76 and shaped to cooperate with and receive a portion of the hemostatic valve 80 seated in the second layer lumen 88.

FIG. 6 illustrates an exploded view of the cover pad 64 of some embodiments of the invention. As shown in this figure, the second layer lumen 88 of the second central layer 96 is larger than the third layer lumen 89 of the third layer 100. This figure also shows that in some embodiments the hemostatic valve 80 is formed by two circular pads 105 and 110. In other embodiments, the hemostatic valve 80 may contain a different number of pads and the pads may be shaped differently.

The circular pads 105 and 110 are formed from a soft rubber material in some embodiments, while they might be formed by other materials in other embodiments. The pads have two slits 115 and 120 at a 90° angle with each other. These two slits allow the guide wire 20 to pass through the central lumen 76. However, the 90° arrangement of the slits plus the composition of the pads 105 and 110 limit the back flow of blood from the central lumen 76. Although the valve 80 is formed by two pads 105 and 110 in some embodiments, one of ordinary skill will realize that the valve 80 is formed differently in other embodiments. (e.g., with different number of pads, different composition for the pads, different shaped pads, different type of valve, etc.)

2. The Stem

As mentioned above, the stem 68 allows the bandage 85, affixed to the stem 68, to be placed in the subcutaneous tissue and within the access tract 48. In some embodiments, the stem 68 is roughly cylindrical in shape with a proximal end and an opposing distal end. In other embodiments, the stem 68 is conical, approximately conical/cylinder, or any other shape. The proximal end is affixed to the cover pad 64. In some embodiments, the distal end cooperates with the bandage 85, to position the bandage subcutaneously within the access tract 48. In different versions of the apparatus 60, the stem 68 may have different lengths, in order to position the bandage 85 at different depths within the access tract 48 based upon patient's circumstances. In some embodiments, the stem may be entirely covered by the bandage. Alternatively, in some embodiments, the stem 68 is a telescopic stem that is capable of achieving different lengths by extending and contracting (i.e., the length of the stem 68 is extended or shortened by telescoping the stem 68). In other embodiments, the stem 68 may be sectioned and joined together, one section at a time, to create an appropriate length for each individual need (i.e., the length of the stem 68 allows adjustments by being sectioned and joined together, one section at a time).

The stem 68 allows the bandage 85 to be placed within the access tract 48 without causing the bandage 85 to flatten near the epidermal layer 44. In so doing, the bandage 85 is placed closer to the vascular puncture 38, thereby reducing the chance of hematoma or other undesirable effects from developing. Section V below elaborates on how different embodiments of the invention deliver a hemostasis bandage (e.g., bandage 85) to different depths within or through the skin.

3. The Bandage

As mentioned above, the bandage 85 is located at the distal end of the stem 68 in some embodiments. The bandage 85 serves to occlude the access tract 48 and provide hemostasis within the access tract 48 without undesirable side effects. In some embodiments, the bandage 85 is a plug that contains a central lumen 76 designed to accept the guide wire 20 and is a component of the delivery hemostatic apparatus 60. As shown in FIGS. 3-6, the bandage 85 in some embodiments has a tapered tip 86 to facilitate its entry into vascular puncture tract 48. A tapered tip (e.g., tip 86) can simplify the bandage's entry into the puncture tract 48 and thereby allows the bandage 85 to be inserted into the puncture tract with or without the use of a wire to guide it. The tapered tip 86 can have an angle that is sharp or mild as described in Section VI below.

The depth at which the bandage 85 is positioned in the access tract 48 will be approximately equal to the length of stem 68. In some embodiments, the diameter of the bandage 85 is approximately equal to the diameter of the access tract 48. The bandage and its delivery mechanism can be customized for different operating environments by varying their attributes (e.g., varying the length of the stem 68, the dimensions of the bandage 85, etc.).

In some embodiments, the bandage 85 may be made from, or coated with, one or more pro-coagulating materials or agents. Pro-coagulating (i.e., coagulating) materials and agents facilitate coagulation and hemostasis. One such pro-coagulation material is Chitosan. By including one or more pro-coagulating materials or agents on or within the bandage 85, hemostasis can be achieved earlier than would otherwise be achievable. By varying the composition of the bandage 85, the hemostasis rate may be controlled or varied to best fit the needs of each individual patient.

Section VI below will further elaborate on the structure and composition of the hemostatic bandage of some embodiments.

B. Method of Use

As discussed previously, removing the access sheath 10 from the access tract 48 at the completion of an intravascular procedure causes the access tract 48 to naturally collapse onto the guide wire 20. Therefore, the hemostatic apparatus 60 should be inserted into the access tract 48 before the surrounding tissue collapses onto the access tract 48. To be most effective, the operator should be able to insert the hemostatic apparatus 60 quickly, easily and efficiently into the access tract 48.

At the conclusion of an intravascular medical procedure, most of the instrumentation used in the procedure is typically removed from the blood vessel and the access tract. For instance, all the instrumentation except the access sheath 10 might be removed from the blood vessel and the access tract 48. Next, a guide wire 20 is re-inserted into the access tract 48 (e.g., re-inserted through the access sheath) and the sheath 10 is then removed.

To insert the hemostatic apparatus 60 into the access tract 48, the apparatus is first threaded onto the guide wire 20 by inserting the side of the guide wire 20 (which protrudes out of the patient) through the hole in the tapered tip 86 of the bandage 85, through the central lumen 76, through the slits 115 and 120 of the pads 105 and 110 of the hemostatic valve 80, and out of all the layers of the cover pad 64. The cover for the adhesive layer 92 of the pad 64 is removed to reveal the adhesive layer 92. Next, the hemostatic apparatus 60 is advanced into the access tract 48 until the bandage 85 is properly placed at the appropriate depth within the puncture tract and the adhesive layer 92 comes in contact with the epidermal layer 44. Different embodiments deliver the hemostatic bandage 85 to different depths within the puncture tract 48. Section V elaborates on how different embodiments of the invention deliver the hemostatic bandage to different depths within or through the skin.

With the adhesive layer exposed, the cover pad 64 can firmly adhere to the epidermal layer 44 to prevent the bandage 85 from moving within the access tract 48 during its application to the patient. With the hemostatic apparatus 60 properly positioned, the guide wire 20 can be removed, as shown in FIG. 4.

With the hemostatic apparatus 60 in place, the hemostatic valve 80 prevents back bleeding through the central lumen 76. The bandage 85 acts to seal the remaining portion of the access tract. By placing the hemostatic apparatus 60 within the access tract 48, the bandage 85 and the cover pad 64 both obstruct the flow of blood from the vascular puncture 38.

In some embodiments, the bandage 85 is coated with, contains or is completely composed of Chitosan or other pro-coagulant (i.e., coagulant) material (i.e., coagulating agents). The use of coagulating agents in the bandage 85 further impedes the blood flow. Section VI further elaborates on the structure and composition of the bandage of some embodiments. Next, the removal of the guide wire 20 causes the access tract 48 to collapse. The tissue surrounding access tract 48 exerts force on the tapered tip 86 of the bandage 85, thereby promoting the collapsing effect, which closes the central lumen 76. The insertion of the bandage, the use of the coagulating agent, and the collapse of the surrounding tissue restrict the flow of blood from the blood vessel 28, thereby quickly and efficiently achieving hemostasis. To achieve hemostasis, a physician might also exert minimal pressure on the cover pad 64 in some cases for a small duration of time (e.g., thirty to sixty seconds). Additionally, the bandage 85 remains in the patient for a suitable amount of time to achieve hemostasis, which can be as little as 2 to 60 minutes, in some cases.

After a suitable period of time to allow for recovery and healing of the patient, the bandage 85 is removed from the living organism by pulling the cover pad away from the patient. After the removal of hemostatic apparatus 60, a light topical dressing may be applied to the wound.

### IV. Radial Artery Hemostatic Bandage

FIGS. 7-11 illustrate another example of a hemostatic apparatus 360 of some embodiments of the invention. The apparatus 360 is designed for a radial artery application. Specifically, after the completion of an operation that uses the radial artery, the hemostatic apparatus 360 wraps around a patient's wrist and occludes a puncture tract created by the operation.

The hemostatic apparatus 360 includes (1) a wristband 364 with an interior surface 350 and an exterior surface 355, (2) a support block 368 affixed to the interior surface 350 of the wristband 364, (3) a bandage (or plug) 385 protruding from the support block 368, (4) a central lumen 376, and (5) two attaching members 397 and 398 at opposing ends and opposing sides of the wristband 364.

Like the plug 85 of apparatus 60, the plug 385 of apparatus 360 is coated with, contains, or is composed entirely of Chitosan or other pro-coagulant material in some embodiments. Also, like the plug 85, the plug 385 in some embodiments can have many shapes, but typically has a shape that facilitates its insertion into the puncture tract. For instance, in some embodiments, the bandage has a tapered tip. Like the plug 85, the plug 385 in some embodiments is positioned within the puncture tract to achieve hemostasis. Other embodiments, however, do not position the plug 385 entirely within the puncture tract. Instead, these embodiments position only the plug's bottom portion within the tract, or position the plug simply over the puncture tract.

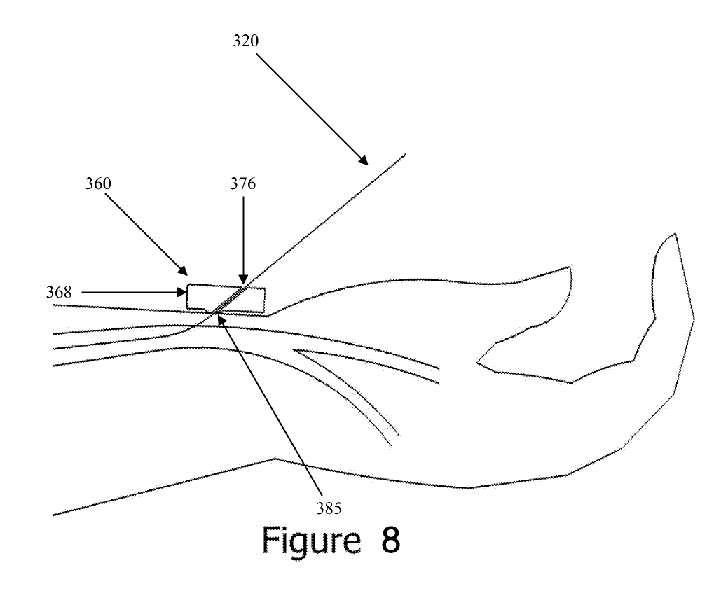

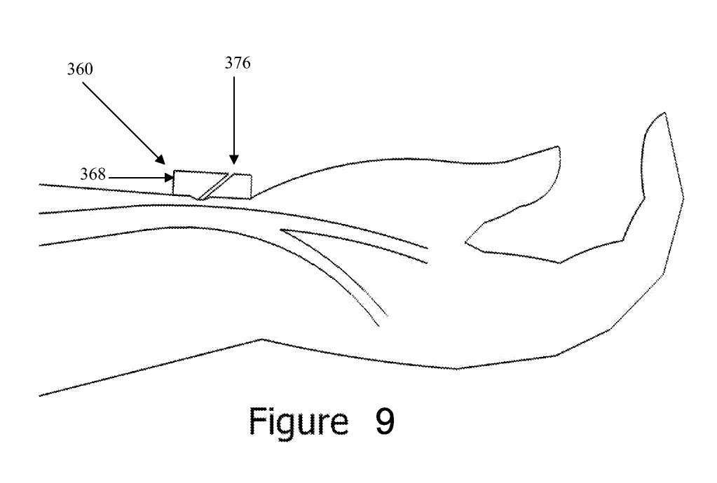

Like the apparatus 60, the apparatus 360 has a central lumen 376 that is used to guide the plug 385 into or over the puncture tract. The central lumen 376 is defined from the outer surface of the wristband 364 through the support block 368 and through the center of the bandage 385, as illustrated in FIGS. 8-9. The central lumen 376 allows a wire to pass through the hemostatic apparatus 360 in order to position the plug 385 into or over the puncture tract as illustrated in FIG. 8. Specifically, after the completion of an operation that uses the radial artery, some embodiments position the apparatus 360 onto the patient by sliding the central lumen 376 over a wire (placed in the puncture tract) until the plug 385 is within or over the puncture tract. As mentioned above and further described below, different embodiments position the bandage 385 in different depths within the puncture tract, while other embodiments simply position the bandage 385 on the patient's skin (i.e., epidermis) over the puncture tract.

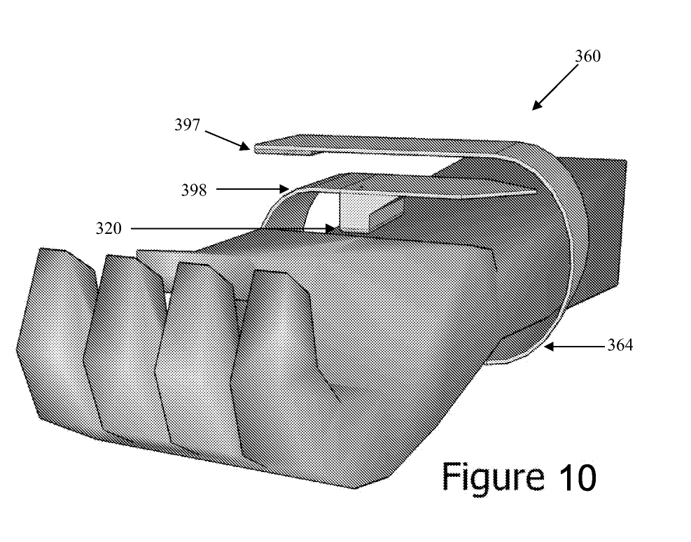

Once the apparatus 360 is positioned onto the patient, the two attaching members 397 and 398 are adjustably attached to secure the wristband on the patient, as illustrated in FIG. 10. In some embodiments, the two attaching members 397 and 398 are made of Velcro or a Velcro like material, so that one member is composed of tiny nylon hooks and the other of loops that interlock with hooks. Other embodiments might use different types of attaching members. For instance, some embodiments might use one or two adhesive layers on opposing ends and sides of the wristband 364 in order to wrap the wristband 364 around the patient's wrist.

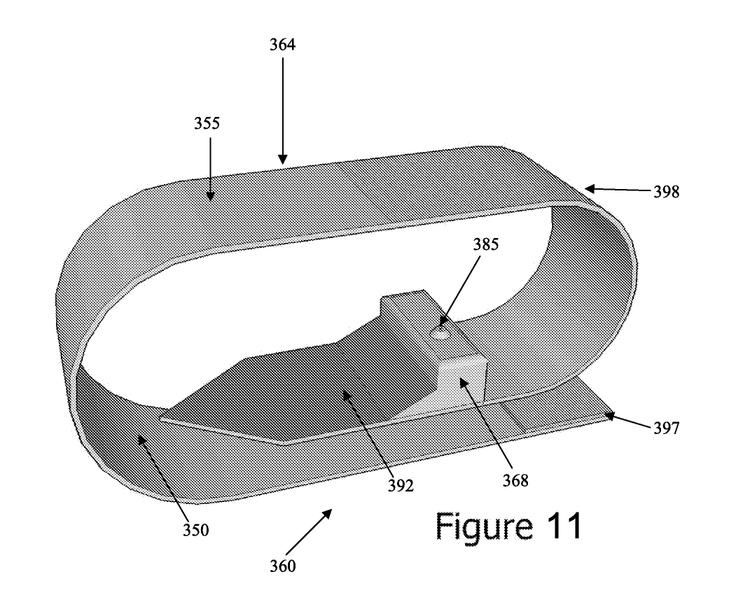

As shown in FIG. 11, an adhesive layer 392 is placed over one end of the interior surface 350 of the wristband 364 in some embodiments. This adhesive layer 392 is covered by a protective strip (not shown) when the apparatus 360 is not deployed. The strip protects the adhesive layer 392 from degradation before the apparatus 360 is deployed. The strip is removed from the adhesive layer 392 when the bandage is being deployed, in order to enable the adhesive layer to affix the apparatus 360 to the patient's skin when the wristband 360 is wrapped around the patient's wrist.

The apparatus 360 of FIGS. 7-11 operates in a similar manner to the apparatus 60 of FIGS. 3-5. Specifically, the bandage 385 of the apparatus 360 is pushed into or over a puncture tract by passing the central lumen 376 of the apparatus 360 over a guide wire 320 that is positioned in the access tract as illustrated in FIG. 8.

Once the bandage 385 has been placed in its desired position upon the tract, the operator pulls the strip off the adhesive layer 392. This exposes the adhesive layer 392 so that the operator can press the adhesive layer 392 onto the skin of the patient's wrist as illustrated in FIG. 10, and thereby affix the wristband 364 securely to the patient's wrist. The operator may then tighten the wristband 364 by pulling the ends of the wristband 364 in opposite directions in such a manner as to encircle the wrist of the patient and overlap the ends of the wristband as illustrated in FIG. 10. Once the wristband 364 has been tightened sufficiently to be secure, the attaching members 397 and 398 are pressed against one another to secure the wristband 364 in place.

Before or after the wristband 364 is affixed to the skin of the patient, the guide wire 320 is removed, and this removal allows the access tract to collapse as illustrated in FIG. 9. Also, the bandage 385 of the apparatus 360 exerts pressure on the access tract to close the tract and secure hemostasis. Accordingly, the insertion of the bandage, the use of the coagulating agent, and the collapse of the tissue restrict the flow of blood from the blood vessel and thereby quickly and efficiently result in hemostasis.

The bandage 385 has to remain affixed to the patient for a suitable amount of time to achieve hemostasis. This amount can be as little as thirty to sixty minutes in some cases. After a suitable period to allow for recovery and healing, the bandage is removed from the living organism by pulling the attaching members 397 and 398 apart and withdrawing the apparatus 360 from the patient. After the removal of the apparatus 360, a light topical dressing might be applied to the wound.

The delivery apparatus 360 and bandage 385 of some embodiments constitute a significant advance in the fields of cardiology, radiology and vascular surgery as it significantly improves upon the prior art by providing an effective means of completely sealing a vascular puncture site, even in anti-coagulated patients, without bleeding and hematoma formation. Compared with the topical application of a bandage as used in the prior art without the precise guide wire directed positioning of the invention's insertion bandage tip, the probability of hematoma formation and the need for prolonged application of external pressure is greatly reduced by using the apparatus 360. The apparatus 360 will reduce patient discomfort, improve sheath related complication rates due to bleeding and hematoma formation, eliminate intra-arterial trauma, reduce hospitalization time and allow rapid mobilization and earlier discharge of patients following catheter based vascular procedures.

While the invention has been described herein with reference to numerous specific details, one of ordinary skill in the art will recognize that the invention can be embodied in forms without departing from the spirit of the invention. For instance, even though several wire-guided devices were described above, one of ordinary skill will realize that some embodiments might not be implemented as wire-guided hemostatic devices. By way of example, the apparatus 360 does not need to be implemented as a wire-guided apparatus 360. Instead, it can be simply secured to the patient by visually placing its plug 385 within or over the puncture tract. In such embodiments, no central lumen 376 needs to be defined in the apparatus 360.

Even though the apparatus 360 is a wristband 364 designed for a radial artery application, other embodiments might implement the hemostatic device differently so that it can affix to other parts (e.g., the leg, stomach, etc.) of the patient's body. For example, in some cases, the invention's bandage will be implemented as an arm band or a thigh band to achieve hemostasis of the brachial artery in the arm or the femoral artery in the thigh. Thus, one of ordinary skill in the art would understand that the invention is not to be limited by the illustrative details contained herein.

V. Delivering the Bandage to Different Depths within or Through the Skin

As mentioned above, different embodiments of the invention deliver the hemostatic bandage to different depths within or though the skin. Before discussing these varying depths, it is helpful to understand the relevant terminology concerning the various layers of skin. Therefore, Section A provides a background discussion of the different layers of skin in a human. Section B then provides different examples for deploying the bandage at different depths within or through the skin.

A. The Skin

The skin can be described in terms of three layers. These three layers are (1) an epidermis layer, (2) a dermis layer, and (3) a subcutaneous tissue layer.

The epidermis layer is the external layer of skin that faces the outside world. The epidermis layer is mainly composed of cells, where each cell type serves a specific barrier function. As a result, the epidermis layer serves as a protective barrier against the external environment. The epidermis layer can be further divided into sub layers of stratum basale, stratum spinosum, stratum granulosum, stratum licidum, and stratum corneum.

The dermis layer is an internal layer coupled to the epidermis layer by a dermal-epidermal junction. The dermal-epidermal junction is a structure that connects together the epidermis and dermis layers. The function of the dermis layer is to provide support and durability to nerves, vasculature, and other structures. The dermis layer resists deformation from outside forces, by returning the skin to its resting state after receiving the outside forces. The dermis layer can include collagen, elastic tissue, and reticular fibers.

The subcutaneous tissue layer is located below the dermis layer. This layer is important for regulating body and skin temperature. The size of this layer varies throughout the body and varies from person to person. The subcutaneous tissue layer includes a layer of fat and connective tissue that houses larger blood vessels and nerves. The third layer is sometimes referred to as hypodermis tissue.

B. Examples of Different Depths within or Through the Skin

The hemostatic bandage of different embodiments (e.g., hemostatic bandage) can be inserted into different depths within or through the skin.

As mentioned above, the epidermis layer is the external layer of skin that faces the outside world. An advantage of delivering a bandage at this depth is that in some embodiments of very superficial wounds with sensitive bleeding vessels primarily just below the epidermis of the skin, the apparatus may deliver its hemostatic bandage just proximal to the sensitive bleeding sources in order to exert maximal hemostatic effect without entering into sensitive bleeding structures.

As mentioned above, the dermis layer is an internal layer of skin that is coupled to the epidermis layer by the dermal-epidermal junction. An advantage of delivering a bandage at this depth is that in some embodiments of superficial wounds with bleeding primarily within the dermis of the skin, the apparatus may deliver its hemostatic bandage directly to the bleeding sources in the dermis.

As mentioned above, the subcutaneous tissue layer is located below the dermis layer. An advantage of delivering a bandage at this depth is that by delivering the active bandage component into the subcutaneous tissue, the bandage can exert its hemostatic action directly on the bleeding source in some embodiments.

In some embodiments, the bandage may be removed from the living organism once adequate hemostasis has been achieved. In this manner, the bandage acts as a disposable hemostatic bandage.

### VI. The Structure and Composition of the Bandage

As mentioned above, the structure and composition of the hemostatic bandage can be different in different embodiments. For instance, the hemostatic bandage can have different shapes in different embodiments. The bandage can also be composed of different materials in different embodiments.

Section A below describes different structures for the hemostatic bandage. Section B then describes different materials that can be used to make the bandage. Section C describes the use of different coagulating materials and agents for making or coating the bandage.

A. The Structure of the Bandage

The hemostatic bandage of some embodiments (e.g., the hemostatic bandage) can have different shapes in different embodiments. For instance, the hemostatic bandage can be conical, cylindrical, approximately conical/cylindrical, or any other shape. In some embodiments, the hemostatic bandage has a tapered tip.

As mentioned above, the tapered tip can be a mild or sharp taper. In some embodiments, the shape of mild tapered tip is cylindrical, bullet shaped, or a blunt rounded tip. In other embodiments, the tapered tip includes any other shape. A tapered tip is especially useful when a guide wire is not used for insertion.

In some embodiments, the hemostatic bandage has a hollow interior that allows the bandage to couple onto a stem. This couple can be strengthened by an adhesive glue. In other embodiments, the hemostatic bandage and stem are not separate components. Instead, the bandage and the stem form an inseparable member/element of the hemostasis apparatus.

In some embodiments, there is a trough that can be located in the cover pad where the cover pad meets the proximal end of the stem. The trough provides an opening for the bandage to couple to or go through. The coupling of the bandage at the trough can be strengthened by using an adhesive material inside the trough.

In some embodiments, an optional guide wire can be inserted through the central lumen. The central lumen is defined as an opening from the outer surface of the center of the cover pad, through the center of the stem, and through the center of the tip of the bandage. The lumen allows observation of ongoing bleeding within a vascular access of puncture tract. In some embodiments, a valve located on the cover pad prevents back bleeding through the central lumen when the valve is closed.

The central lumen also gives the physician the choice to use a guide wire to facilitate insertion. The lumen can act as a guide to insert the bandage into a vascular puncture tract by passing a wire through the bandage and the delivery mechanism.

In some embodiments, the bandage is the stem itself (i.e., the stem and the bandage are one component). In some embodiments, this stem/bandage has many of the attributes of the bandage described above and below in Sections III-VI. For instance, it can be made of the same material (e.g., Chitosan, Fibrinogen, etc.), it can have a tapered tip, etc.

B. Composition

Different materials can be used to make the bandage. In some embodiments, the hemostatic bandage is made of a solid flexible material that allows the bandage to bend when the bandage is inserted into a puncture tract. In some embodiments, the bandage is composed of a solid flexible material (e.g., foam). This flexibility reduces the patient's discomfort.

In some embodiments, the hemostatic bandage is made from one or more absorbent materials. For instance, the hemostatic bandage can be composed of absorbent materials such as polymeric foam, polyurethane, hydrophilic polyurethane, etc. In some embodiments, polymeric foam is a hydrophilic polyurethane foam.

In some embodiments, the hemostatic bandage may be composed of absorbent cotton, cotton wool, or cotton gauze. In other embodiments, the hemostatic bandage may be composed entirely of Chitosan. In some embodiments, the Chitosan can be a lyophilized solution molded (i.e. shaped) to the bandage configuration.

C. Coagulating Materials

In some embodiments, the hemostatic bandage may be made from, contain, or be coated with one or more pro-coagulating (i.e., coagulating) materials (i.e. coagulating agents). Coagulating materials facilitate coagulation and hemostasis. Examples of coagulating materials that can be used include Chitosan, Fibrinogen, Thrombim, self-assembling peptides, and other types of coagulating materials.

In some embodiments, the bandage is coated with, contains, or is completely composed of Chitosan or other pro-coagulant (i.e., coagulant) material (i.e. coagulating agents). The use of coagulating agents in the bandage further impedes the blood flow. Section VI further elaborates on the structure and composition of the bandage of some embodiments.

Coagulating materials can also include solid materials such as polyurethane and hydrophilic polyurethane. Some embodiments combine on or more coagulating materials with one or more absorbent materials to form their respective hemostatic bandage. Other embodiments coat the absorbent material of a hemostatic bandage with coagulating materials.

For example, Chitosan in an acidic solution can be incorporated on or into a foam bandage by means of dipping or soaking the bandage. In some embodiments, the foam bandage is lyophilized (e.g., cooled so that the coagulating agents crystallize or otherwise solidify) after dipping the bandage into the solution.

The use of coagulating agents in the bandage causes coagulation in the puncture tract, which further impedes the blood flow. As a result, hemostasis is achieved earlier than otherwise possible. By varying the composition of the bandage, the hemostasis rate may be controlled or varied to best fit the needs of each individual patient.

The delivery apparatus and bandage of some embodiments constitute a significant advance in the fields of cardiology, radiology, and vascular surgery as it significantly improves upon the art by providing an effective means of completely sealing a vascular access puncture site, even in anti-coagulated patients, without bleeding and hematoma formation. Compared with the topical application of a bandage as used in the prior art without the precise guide wire directed positioning of the invention's insertion bandage tip, the probability of hematoma formation and the need for prolonged application of external pressure is greatly reduced by using the apparatus. The apparatus will reduce patient discomfort, improve sheath related complication rates due to bleeding and hematoma formation, eliminate intra-arterial trauma, reduce hospitalization time and allow rapid mobilization and earlier discharge of patients following catheter based vascular procedures.

While the invention has been described herein with reference to numerous specific details, one of ordinary skill in the art will recognize that the invention can be embodied in forms without departing from the spirit of the invention. For instance, the examples above describe a wire guided implementation. In other embodiments, the apparatus may be inserted without a guide wire. Several such embodiments are described in U.S. Provisional Patent Application 60/863,565, which is incorporated herein by reference.

Some embodiments of the invention provide an apparatus for achieving hemostasis in a puncture tract in a patient (e.g., a human or animal) created during a medical procedure or operation. In other embodiments, the tract might be a result of a traumatic injury (e.g., injury that occurred outside of a hospital) that created a traumatic wound, such as a bullet wound, shrapnel or knife puncture. In some embodiments, the bandage (i.e., plug) is part of an adjustable strap that is used to secure the bandage within the puncture tract and maintain pressure until hemostasis has been achieved. For instance, some embodiments are designed for a radial artery application. In some of these embodiments, the adjustable strap is an adhesive wristband that is wrapped around the patient's wrist, after the completion of an operation that uses the radial artery (which is an artery that passes through the patient's wrist). Other embodiments might implement the hemostatic device differently so that it can affix to other parts (e.g., the leg, stomach, etc.) of the patient's body.

Some embodiments are described above with reference to a human patient. However, as mentioned above, the invention can be used in any puncture tract for other living organisms. Thus, one of ordinary skill in the art would understand that the invention is not to be limited by the illustrative details contained herein, but rather is to be defined by the appended claims.

What is claimed is:

1. An apparatus comprising:
a. a bandage for inserting into a puncture tract to achieve hemostasis; and
b. a strap coupled to the bandage, wherein the strap is for maintaining the bandage on a part of a patient.

2. The apparatus of claim 1, wherein the strap is for at least partially wrapping around the part of the body.

3. The apparatus of claim 2, wherein a first portion of the strap overlaps with a second portion of the strap.

4. The apparatus of claim 1, wherein the puncture tract is created during a medical procedure.

5. The apparatus of claim 4, wherein the medical procedure is a catheter-based intravascular procedure.

6. The apparatus of claim 1, wherein the bandage is for maintaining on the part of the body until hemostasis is achieved.

7. The apparatus of claim 6, wherein the bandage is maintained at a particular pressure by the strap.

8. The apparatus of claim 1, wherein the strap is adjustable.

9. The apparatus of claim 1, wherein the strap is a wristband.

10. The apparatus of claim 1, wherein the bandage is a plug.

11. The apparatus of claim 10, wherein the plug is for inserting at different depths of the puncture tract.

12. The apparatus of claim 10, wherein the plug is wire guided into the puncture tract.

13. The apparatus of claim 1, wherein the bandage comprises a material that promotes coagulation.

14. The apparatus of claim 13, wherein the material is Chitosan.

15. The apparatus of claim 1, wherein the bandage is for achieving hemostasis of a radial artery in a wrist.

16. The apparatus of claim 1, wherein the bandage is for achieving hemostasis of a brachial artery in an arm.

17. An apparatus comprising:
a. a bandage for applying over a puncture tract to achieve hemostasis; and
b. a strap coupled to the bandage, wherein the strap is for maintaining the bandage on a part of a patient.

18. A method for achieving hemostasis, the method comprising:
a. inserting a bandage into a puncture tract; and
b. using a strap to maintain the bandage on a part of a patient comprising the puncture tract.

19. The method of claim 18 further comprising removing the bandage from the puncture tract after hemostasis has been achieved.

20. The method of claim 18, wherein the strap is at least partially wrapped around the part of the body.

21. The method of claim 18, wherein the puncture tract is created during a catheter-based intravascular medical procedure.

22. The method of claim 18, wherein the bandage is a plug, wherein a portion of the plug is inserted into the puncture tract.

23. The method of claim 18, wherein the bandage is a plug, wherein the plug is inserted into the puncture tract by using a guide wire.

24. The method of claim 18, wherein the bandage comprises a material that promotes coagulation.

25. A method for achieving hemostasis, the method comprising:
a. applying a bandage over a puncture tract; and
b. using a strap to maintain the bandage on a part of a patient comprising the puncture tract.
